(19) United States
(12) Patent Application Publication
(10) Pub. No.: US 2013/0203077 A1
(43) Pub. Date: Aug. 8, 2013

(54) QUANTITATION OF URINARY TISSUE FACTOR FOR THE DIAGNOSIS AND SCREENING OF CANCER

(71) Applicants: Thomas Mole Herd, Eastleigh (GB); Robert Greenfield, Hartsdale, NY (US)

(72) Inventors: Thomas Mole Herd, Eastleigh (GB); Robert Greenfield, Hartsdale, NY (US)

(73) Assignee: C-TERM DIAGNOSTICS LIMITED, Sutton Coldfield (GB)

(21) Appl. No.: 13/879,687

(22) PCT Filed: Oct. 19, 2011
(86) PCT No.: PCT/GB2011/052026

§ 371 Date: Apr. 16, 2013

(30) Foreign Application Priority Data
Oct. 19, 2010 (GB) 1017615.4

(51) Int. Cl. G01N 33/574

(52) U.S. Cl. CPC G01N 33/574
USPC 435/7.9; 436/501

(57) ABSTRACT

We provide a novel technological approach for quantitation of c-terminal fragment of uTF for the purpose of quantitation, diagnosis and population screening of cancer. We provide a rationale for measuring c-terminal fragment, methods for making assays, monoclonal and polyclonal antibodies, together with accepted methods of sample preparation. Taken together these proposals and ideas constitute a novel approach for diagnosis and population screening for cancers using urine from cancer patients. In addition we believe that our approach may be useful in the diagnosis of other pathological conditions.

### TISSUE FACTOR

A series of coordinated reactions takes place in the body whenever blood clots. The major physiological initiator of these reactions is a membrane-bound glycoprotein known as Tissue Factor (TF), which is normally separated from the blood stream by the vascular endothelium. Bleeding, caused by injury or tissue damage, activates a complex enzyme cascade as TF becomes exposed to the bloodstream.

However, in disease states, leucocytes or the vascular endothelium may abnormally express to cause intravascular coagulation. The blood coagulation cascade is also relevant to diseases such as haemophilia in which sufferers are deficient in blood proteins necessary for normal clotting, and is linked to vascular diseases such as heart attacks and strokes in which clotting may lead to occlusion of blood vessels.

Coagulation is also activated in inflammatory illnesses and cancer. This document discusses the role that Tissue Factor plays in these processes and also the highly significant part it plays in angiogenesis, cancer, metastasis, inflammation, drug resistance, fertility and wound healing. Refer to FIG. 1.

### Forms of Tissue Factor

Two forms have been described: free TF (which includes a soluble, alternatively spliced and bloodborne TF) and membrane-bound TF. Soluble TF results from proteolytic cleavage at or near the linkage between the transmembrane and the extracellular domains of the TF molecule, forming protein fragments, whereas the other variants of free TF results from alternative splicing of the primary RNA transcript (Bogdanov et al., 2003; Bogdanov et al., 2006; Guo et al., 2001). These free forms of TF circulate in the plasma and are biologically active. Plasma TF can also be found circulating in association with cell-derived membrane microparticles. TF-bearing microparticles, which arise mainly from monocyte-macrophage membrane-lipid rafts or from regions of high raft content; in this case TF is called bloodborne. The circulation TF microparticles alone do not seem to effectively initiate coagulation, but when they bind and fuse with activated platelets via the mechanism involving P-selectin glycoprotein ligand-1 on the microparticles and P-selectin on the platelets, they initiate coagulation (del Conde, Shrimpton, Thiagarjan & Lopez, 2005).

The membrane-bound forms of TF include both cellular TF (such as that found on monocytes, macrophages, endothelial and tumor cells) and lipid-vesicle-bound TF in urine [microparticles, exosomes] or in semen [prostasomes]. Cellular TF is found in three pools: surface, encrypted and Intracellular. On the plasma membrane of cells, TF resides mostly in a cryptic configuration; its release, which is often referred to as de-encryption, coincides with an increase in cell-surface phosphatidylserine, resulting in apoptosis and necrosis.

### Expression of TF

TF is a constituent of both the subendothelial layer of the vascular well and the extravascular tissue. It thereby forms a protective lining around the blood vessels and is ready to activate blood coagulation if vascular integrity is compromised (Ryan et al., 1992). Endothelial cells and blood monocytes (in contact with the bloodstream) do not constitutively express TF and do not have stores of TF.

Although TF is not normally expressed by cells within the bloodstream, gene transcription and subsequent protein expression can be induced in monocytes, macrophages and endothelial cells by thrombin, endogenous inflammatory mediators such as interleukins 1& 6, tumor-necrosis factor—alpha (TNF-α), vascular permeability factor (VPF), complement C5a, phorbol esters, plasma lipoproteins, plasma protein, collagen, Immune complexes and microorganisms as well as other physiological and pathological mediators (Amiridrosravi et al., 1996; Østerud, Olsen & Wilsgard, 1990; Roth, 1994).)

Platelets and lymphocytes can induce de novo TF biosynthesis in monocytes and endothelial cells. Likewise monocytes and natural killer cells can upregulate TF expression in endothelial cells (Napoleone, Di Santo & Lorenzet, 1997). This cell-cell cooperation is essential for TF induction in inflammatory disease and in the cell mediated immune response. Shearing forces and hypoxia also induce TF expression in many cells and provoke coagulation. TF activity also relies on changes in cellular phospholipid composition).

Urine is known to possess powerful ‘procoagulant activity’ [PCA] (Grunke et al, 1835) that normalizes the clotting time of haemophiliac patients in vivo (von Kaula et al, 1954). von Kaulla noted that preparations obtained by adsorbing human urine with barium sulphate were largely associated with materials that normalized the clotting of haemophiliac plasma in vitro (von Kaula et al, 1965). It was demonstrated that minute amounts of these preparations had the ability to correct the clotting of patients who have a high titre of circulation ‘antithromboplastin’, now known as tissue factor pathway inhibitor (TFPI) (von Kaula et al, 1963). The PCA was first thought to be a tissue thromboplastin-related substance. It was later found that urinary PCA catalysed the conversion of prothrombin to thrombin only in the presence of platelet factor 3, factor V and Ca²⁺—a prothrombinase type of activity, and was therefore classified as a ‘platelet cofactor’ rather than TF (Matsimura et al, 1970, Caldwell et al, 1963). Aoki and von Kaulla further investigated the interaction between platelet factor 3, factor V, prothrombin, and urinary PCA. They concluded that platelet factor 3 could be replaced by phospholipids at the activation of prothrombin with partially purified urinary procoagulent (uPC), provided that the lipid was used under optimal conditions. A similar observation was made by Joist and Alldaersig, who noted that phospholipid or platelets merely enhanced urinary PCA in the coagulation of plasma (Joist et al, 1967). Subsequently, a factor that had the properties of TF was described in urine. Kurosawa et al. purified the uPC by chromatography on phenyl-sepharose and showed that it promoted clot formation in a factor VII-dependent manner (Kurosawa at al, 1984). Aoki and von Kaulla suggested that the urinary PCA existed as small particles and they reported a factor with the properties of ‘prothrombinase’. Subsequently, it was demonstrated that urinary PCA existed in lipid-associated vesicles and was mainly factor VII-dependent (Wiggins et al, 1987). The uPC can activate factor X in the prescense of factor VII and calcium and was inhibited by concanavalin A (Zacharaski et al, 1974). Activity was restored by the addition of β-methyl-glucoside (Kurosawa et al, 1984). Later, it was confirmed that the procoagulant was TF by demonstrating almost total inhibition of the activity in the presence of a specific antibody to human TF (Carty et al, 1990). The activity appeared to be associated with membrane vesicles that passed through a 220 nM filter, but not one with 100 nM pores.

Following purification and the removal of contaminant Tamm-Horsfall mucoprotein, the procoagulant was found to exist in micro-aggregates. These micro-aggregates were composed of basic functional units of 151 kb consisting of two sub-units of 68 and 76 kD held together by disulphide bonds (Kurosawa et al, 1984). This contrasts with an established molecular weight for human TF of 43 kD. These differences probably reflect the relative impurity of the former preparation, since the mature TF mRMA transcript is the same in a variety of human tissues, including kidney. Indeed, a subsequent estimate of uTF using western blotting revealed that uTF has a similar molecular weight to TF derived from other human tissues [43 kD] (Carty et al, 1990). The vesicles can be sedimented by ultracentrifugation (Carty 1990) and TF activity can be recovered by solubilizing the TF-containing pellet in the detergent β-octyl-glucopyranoside (Lwaleed et al, 1999). The vesicles seemed to be intimately associated with fibrin strands when the latter were generated in vitro (Carty 1990) and showed specific binding to gold-labelled marine anti-TF (Lwaleed et al 1998). The labelling of these vesicles with gold-labelled annexin V (a placental anti-coagulant protein-I) showed that they contained not only TF, but also anionic phospholipids (Lwaleed et al, 1998), which could be the lipid provider for uTF activity. Indeed, uTF activity was inhibited in a dose-dependent manner when it was incubated with different concentrations of recombinant annexin V. This result agrees with those obtained for monocyte TF activity (Salta et al, 1994 and Carvalho et al, 1995).

Sources of Urinary TF

The precise source and the underlying biological mechanisms of uTF elevation in disease states remain poorly understood. Several studies have attempted to focus on this issue with different results. The demonstration that renal pelvis urine has normal procoagulant content excluded the lower urinary tract as a possible source (Matsumura et al, 1968). Two other possible sources of the uPC are implicated; uPC could be a tissue (excretory) product which passes through the glomerular filter, or be a secretory product of the kidney which originates in renal parenchymal tissue. uTF has a molecular weight of 43 kD and only a weak correlation has been observed between uTF levels and urinary protein excretion and markers of glomerular filtration rate (Lwaleed et al, 1998). Thus, uTF may not be blood-derived. In addition, uTF activity is not affected in clinical states such as haemophilia, heparin treatment and patients with circulating antithromboplastin. The kidney therefore remains a possible source of uPC. Several histochemical and immunolocalization studies have addressed the issue of the renal cell of origin of uTF. Some groups reported localization to the glomerulus (Bukovsky et al, 1992), while others reported signal within renal tubules (Lwaleed et al, 1998). The argument that the procoagulant is produced by renal tubules is supported by clinical observation and experimental models (Matsumura and von Kaulla, 1968; Matsumura et al, 1968; Matsuda et al, 1979; Lwaleed et al, 2007). We found, using a murine monoclonal antibody to human TF, that tubular cells showed a positive signal (Lwaleed et al, 1999). Tubular cells were also positive for annexin V (Lwaleed et al, 1999). It is also possible that renal macrophages and/or tubular calls become activated and secrete an increased amount of TF, particularly in disease states such as malignancy and certain types of inflammatory conditions (Carty et al, 1990). Since uTF elevation is similar in magnitude to that described for monocyte TF, it is reasonable to suggest that uTF levels may reflect the state of monocyte/macrophage ‘activation’ in disease states. A moderate association has been demonstrated between uTF and monocyte TF activity, especially with that obtained with lipopolysaccharide-stimulated cells (Lwaleed et al, 1998), but the stimulus for, and control of uTF production remains to be established.

### uTF and Cancer

Simple tests for biomarker substances in the urine of patients with cancer are an attractive proposition (Wajsman et al (1975). uTF may be such a biomarker in certain circumstances (Carty, 1990, Adamson 1992 & 1993, Lwaleed 1996 & 1997, Adamson 1992, Colluci, 1991 are examples). Although the mechanisms of uTF elevation in cancer patients are poorly understood, its level may be of clinical importance, Carty et al, reported increased uTF activity in patients with colorectal cancer, inflammatory bowel disease, and breast cancer. Although patients with benign colorectal and breast disease also had increased levels compared with normal controls or patients with rheumatoid arthritis, these were less than those in the malignant group. In patients with an abnormal colonoscopy 88 percent had abnormal uTF levels, compared with 24 percent of patients with normal colonoscopy. Adamson et al, showed higher levels of uTP in patients with malignant disease of the bladder and prostate than in controls and patients with benign prostatic hypertrophy. Lwaleed et al. showed no significant difference between normals, surgical controls or patients with benign (non-inflammatory) conditions. However, the controls showed significantly lower uTF levels then those with malignant or benign (inflammatory) conditions. Spillert and Lazaro, Østerud et al., and Edwards et al. suggested that inflammatory conditions interfere with the host immune response, which, leads to an increase in monocyte TF expression, a mechanism which could similarly explain uTF elevation in these conditions. However, Carty et al. found that subjects with rheumatoid arthritis, all of whom had active disease, showed generally normal uTF levels. This suggests that uTF does not behave simply as an acute phase reactant.

uTF activity has shown some correlation to histological tumour grading and/or staging (breast, bladder, prostate, and colorectal cancer), bone scan status (prostate cancer), and serum prostate-specific antigen levels (prostate cancer) (Lwaleed, 1997). Patients with recurrent bladder malignancy also showed higher uTF levels than those with a normal check cystoscopy and levels were higher in patients who subsequently died (Adamson, 1992, Lwaleed, 1998).

### Structure of uTF

All full length membrane-bound tissue factors including uTF are single chain glycoproteins comprising 263 amino acid residues with molecular weight of 43 to 45 kD. They have three domains: 1) an extracellular domain; 2) a transmembrane domain; and 3) a C-terminal cytoplasmic intracellular domain (see FIG. 2).

Each domain provides a critical function essential for the biology of uTF. The extracellular domain (aa 33-251) is responsible far binding to various proteins in the coagulation system such as Factor VII, TFPI and Factor X. The transmembrane domain (aa 251-274) is highly hydrophobic and anchors TF in the plasma membrane of cells. The intracellular domain (aa275-295) mediates signal transduction activity which leads to the activation and inactivation of various genes. The signal transduction function of tissue is vitally important in carcinogenesis. The expression of tissue in cancer cells is known to mediate the expression of VEGF and Integrins. These mediators are important for angiogenesis, cell motility and metastasis. TF plays a role in tumour biology, including growth and spread. Activation of TF in malignant and certain inflammatory conditions may be reflected in its levels in a variety of body fluids. uTF levels are not significantly affected by age, sex or cigarette smoking (Lwaleed et al, 1999) which means that the number of false positive results will be low when screening large populations. Thus, uTF may be potentially useful in monitoring patients with renal disease and cancer.

### Activity Assays.

Advances in the understanding of the molecular structure and the biochemistry of TF have led to the development of assays for measuring various structural components of TF. Different methods have been used to assess TF at the cellular and plasmatic levels, and in other body fluids, with substantial clinical potential. A number of studies have employed assays that rely on the measurement of the procoagulant activity of uTF. These assays also only measure fully functional uTF and focus on the extracellular domain of uTF. One-stage and two-stage kinetic chromogenic assays (KCA) have been used to measure uTF procoagulant activity in disease states (Carty et al, 1990; Lwaleed et al, 1999), glomerulonephritis (Lwaleed et al, 1997a) and solid tumours (Lwaleed et al, 1997b). The effect of renal function on uTF measurements was studied by Lwaleed et al. (1998).

Changes in uTF activity have been demonstrated in several disease states. The uTF PCA, on the other hand, was slightly raised in patients with hyperthyroidism and was significantly increased in patients with Glomerulonephritis and cancer (Carty, 1990; Adamson 1992 & 1993; Colluci, 1991; Lwaleed 1996 & 1997, are examples). The uTF procoagulant activity was normal thromboembolic and liver disease and markedly decreased in patients with parenchymal renal diseases and diabetes mellitus (Qi H et al, 1996).

The KCA assays are manual assays more suitable for research applications but are less suitable for large scale clinical use and for instrument-based applications. Assays that measure uTF “activity” may not reflect the total amount of uTF present in a sample because some of the uTF may be structurally inactive or proteolytically cleaved. These forms will not be measured in functional assays. Functional assays also preferentially measure the extracellular domain.

### Immunoassays.

An immunoassay is more suitable to clinical laboratories and a uTF-specific immunoassay would be most preferable for mutation screening. TF has been measured in urine and other body fluids using immunoassays such as ELISA formats (Fareed et al, 1995). Immunoassays utilize antibodies against the tissue factor protein to measure TF antigen or protein levels rather than the biological activity of TF. Most TF ELISAs in the past have used antibodies directed against the extracellular domain (e.g. American Diagnostica Inc's Imubind Tissue Factor ELISA (REF 845). The extracellular domain is mainly responsible for the procoagulant activity of TF. Such ELISAs can measure various forms of TF including full length TF, “soluble TF” which is formed by the proteolytic cleavage of the extracellular domain of TF and various other fragments that are produced by further proteolysis of TF. The presence of proteolytic fragments of TF in a sample would interfere with the measurement of full length TF (i.e. uTF) because the proteolytic fragments would block the binding of antibodies to the full length antibody. Other forms of TF (i.e. alternately spliced TFs) which also have a native extracellular domain would also interfere with the measurement of uTF for similar reasons.

Thus, to accurately measure uTF antigen in the presence of different forms of TF and proteolytic fragments requires a different approach to the immunoassay. More specifically it requires a novel set of antibodies that will distinguish the uTF from the other TFs.

### Novel Approach for Measuring uTF

uTF like all proteins is synthesized in the cell from N-terminal to C-terminal. Therefore, the presence of the C-terminal end of the uTF indicates that the whole molecule from N-terminal c-terminal has been synthesized. Thus, the C-terminal region of uTF, whether present intact with the whole uTF molecule or as a proteolytic fragment is a good indicator that the whole uTF molecule was synthesize. The c-terminal would also represent only the native uTF molecule and not the alternately spliced variants. Measuring the c-terminal region of uTF should specifically reflect the concentration of the whole uTF molecule and not the spliced variants.

We propose that the measurement of the c-terminal fragment of uTF would be a novel approach for accurately and specifically measuring the concentration of uTF in urine and other biological fluids. The measurement of the c-terminal region of uTF can be accomplished using various quantitative and qualitative immunoassay technologies including ELISA, latex agglutination assays, lateral flew technologies, etc. The important common feature of these assays are the development of polyclonal and monoclonal antibodies, antibody fragments, genetically engineered antibodies and/or other binding proteins that specifically target the c-terminal peptide. We propose that a specific immunoassay/assay using antibodies and specific binding molecules against the c-terminal peptide of uTF would be useful for the specific and accurate quantitation of uTF in biological fluids.

Carson and Yoder (Blood Coagul Fibrinolysis (1992) 3: 779-787) teach that monoclonal antibodies can be developed against the c-terminal fragment or TF. The utilized the monoclonal antibodies to show that the c-terminal peptide can be proteolytically cleaved from TF. They also used the antibodies to characterize the distribution of vesicles containing TF and the orientation of TF within the vesicles. They do not teach that c-terminal antibodies can be used for the quantitative or semi-quantitative measurement of the levels of TF in biological fluids. They also do not teach how to create antibodies to c-terminal TF peptide for use in a two site ELISA or other immunoassay format that can be used to develop an immunoassay for measuring TF.

We propose the following methods for creating anti-c-terminal antibodies that can be used for developing two site ELISA for measuring uTF.

### Monoclonal Antibodies.

The c-terminal peptide of uTF is the 20 amino acid portion of the uTF protein that is below the plasma membrane and sticks out of the plasma membrane into the cytoplasmic portion of the cell. The key role of the c-terminal peptide is to mediate signal transduction as discussed above. We propose that two fragments of the c-terminal region (for example aa 275-285 and aa 286-295) be synthesized by standard methods. Polyclonal and monoclonal antibodies to each of the peptides are made using standard methodologies. Two-site immunoassays are developed using pairs of the antibodies to the peptides. We require that the antibodies to each of the peptides does not block each other when the intact uTF molecule is present in the biological fluid or if the c-terminal 20 amino fragment is present without the rest of the uTF molecule.

Either of the two antibodies can be used as the capture antibody for the ELISA or the detection antibody for the ELISA. Refer to FIG. 3.

### Polyclonal Antibodies.

It is also possible to achieve a two site anti-c-terminal ELISA using a polyclonal antibody. The requirements are that the polyclonal antibody contain antibodies to different regions of the 20-amino acid fragment such that they do not block each other. The polyclonal antibody can be produced by immunizing animals (rabbits, sheep goats) with the entire 20 amino acid c-terminal fragment. Antibodies should be able to be developed to different regions of the 20-amino acid fragment. The polyclonal serum or IgG can be used as the capture and the detection portion of the ELISA.

### Sample Preparation

### Solubilization.

The uTF sits within a liposomal-like microparticle as depicted in FIG. 4. In such a structure the c-terminal portion of the uTF molecule is inside the vesicle (Lwaleed et al 1992). It will be difficult for an antibody to bind to the c-terminal portion or uTF if it is within the vesicle. For measuring the c-terminal fragment of uTF in our proponed assay we propose to incorporate a step whereby the microvesicle or liposomal-like membranes which the uTF molecule site in is solubilized by the addition of a detergent such as Triton X-100, NP-40, deoxycholate or tween 20. Solubilization of the microparticle by detergent will dissolve the microparticle structure and reveal the c-terminal fragment so that the antibodies can bind to the c-terminal fragment.

### Centrifugation.

Another sample preparation technique that can be used is to concentrate the uTF microparticles before the immunoassay is performed. Microparticles are able to be separated from solution by high speed centrifugation. Typical g-forces to accomplish this is 10-20,000 xg which can be performed in a standard laboratory microcentrifuge. Thus, it is possible to concentrate the microparticles by taking a urine sample of for example 1 and centrifuging at 12,000 x g to remove the microparticles from the urine solution. The pelleted microparticles containing uTF can then be solubilized in a small volume of detergent containing buffer (i.e. 50 μl). This procedure will concentrate the particles by 20-fold than what present in urine.

### Preliminary Proof of Principle Studies.

We tried to use a commercial ELISA for tissue factor by American Diagnostice (ref 845) to measure tissue factor levels in urine. This ELISA uses antibodies against the extracellular portion of TF for both capture and detection. We were not able to measure TF in urine using this commercial assay. These findings suggest that a different combination of antibodies are needed to detect TF in urine.

We have performed preliminary studies using ELISA technology to demonstrate the proof of principle that antibodies to the c-terminal peptide of uTF can be useful quantitation of uTF in urine of cancer patients. In this study we used a polyclonal antibody to c-terminal fragment of tissue factor as the capture antibody in an ELISA. The detection antibody in this study was a monoclonal antibody against the extracellular domain. We believe that the ELISA assay is more sensitive in determining differences between malignant and inflammatory conditions. Urines were obtained from commercial sources from patients with previously diagnosed cancers of various types. Urine samples 1.5 ml was centrifuged in microcentrifuge and the pelleted microparticles were re-suspended in a detergent containing buffer. The dissolved microparticle solution was run in the ELISA. FIG. 5 shows that higher concentrations of c-terminal peptide contain uTF was present in urines from lung, bladder, prostate colon, renal lymphoma and skin cancers. Normal (non-cancerous) patients and patients with prostatitis (an inflammatory condition of the prostate) had low levels of uTF.

### Potential Methods to Optimize Quantitation of uTF

The quantitation of uTF may still be optimized by a more sensitive ELISA. This may be accomplished by using a different pair of antibodies than in FIG. 5. A better ELISA may be constructed using two high affinity monoclonal and/or polyclonal antibodies with high specificity directed against different regions of the c-terminal fragment of uTF.

Another improvement in the measuring of uTF may be gained by using a “normalization” factor in urine. For example, it may be useful to normalize the quantity of uTF using the amount of protein in the urine sample. The protein in the sample can be measured using standard assays (BCA, coomassie blue reagent). The amount of uTF determined by ELISA divided by the amount of protein may provide a better metric to discriminate urines derived from patients with or without cancer. Another possible method to “normalize” the uTF amount is to determine the ration of uTF activity divided by the uTF protein content. The ratio of activity/protein may provide a better discriminator of patient with cancer vs non-cancer conditions. Activity for uTF can be measured by any of the methods described above.

### SUMMARY

We provide a novel technological approach for quantitation of c-terminal fragment of uTF for the purpose of quantitation, diagnosis and population screening of cancer. We provide a rationale for measuring c-terminal fragment, methods for making assays, monoclonal and polyclonal antibodies, together with accepted methods of sample preparation. Taken together the proposals and ideas constitute a novel approach for diagnosis and population screening for cancers using urine from cancer patients. In addition we believe that our approach may be useful in the diagnosis of other pathological conditions.

### LIST OF FIGURES

FIG. 1 Cellular tissue initiated coagulation

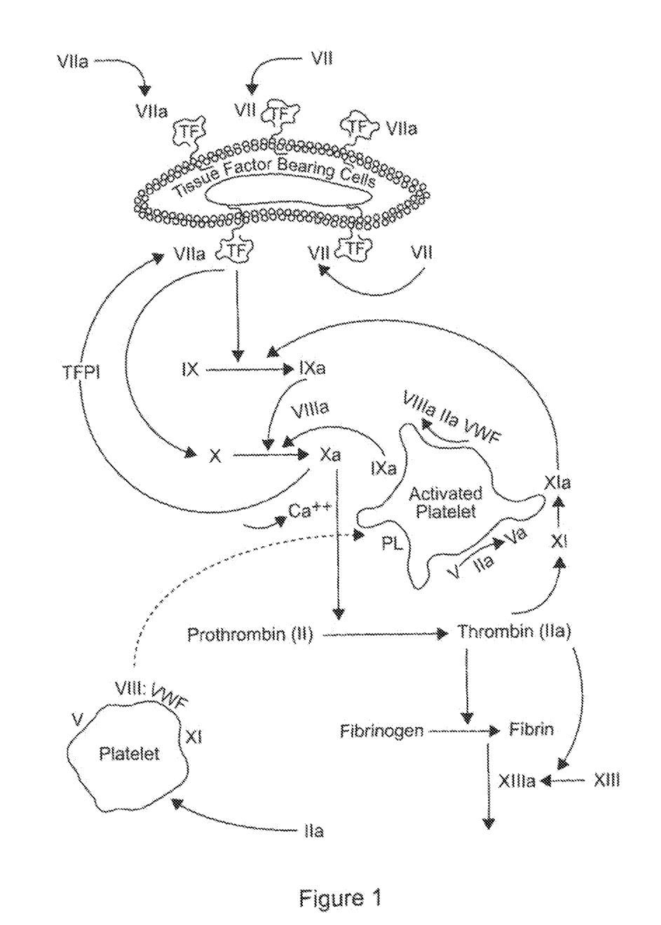

Factor VII binds to TF on TF-bearing cells and is auto-activated to Vila. The resulting complex activates Factor X and Factor IX and a small amount of Thrombin IIa. A feedback mechanism (TFPI) inhibits the TF: Vila complex. However the small amount of thrombin generated activates platelets and Factor V, releases and activates Factor VIII from being bound to von Willebrond factor and activates Factor XI. Activated Factor IX binds to the activated platelets and activates Factor X. Platelet generated Factor X then generates large amounts of Thrombin which is needed for the clot to form.

FIG. 2—A model of TF showing a representation of the three domains.

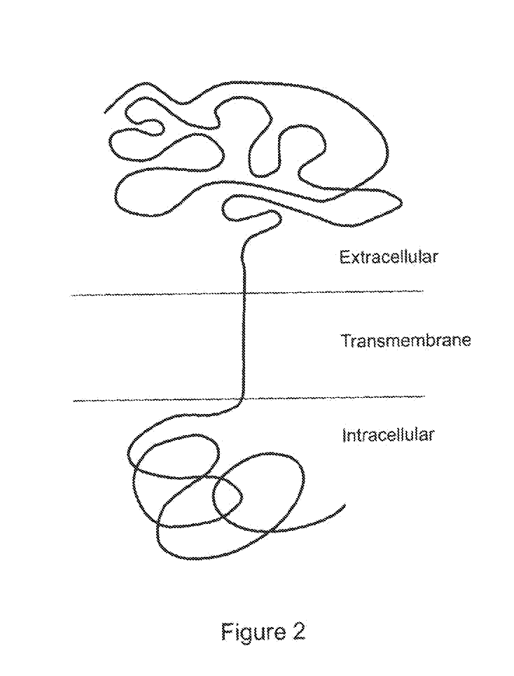

FIG. 3 Showing two antibodies

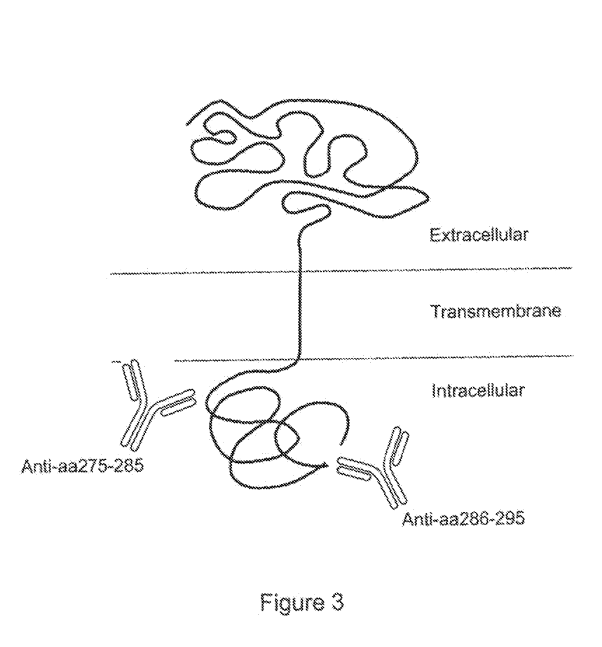

FIG. 4 From: Hugel et al. Physiology (2005) 20:22-27.

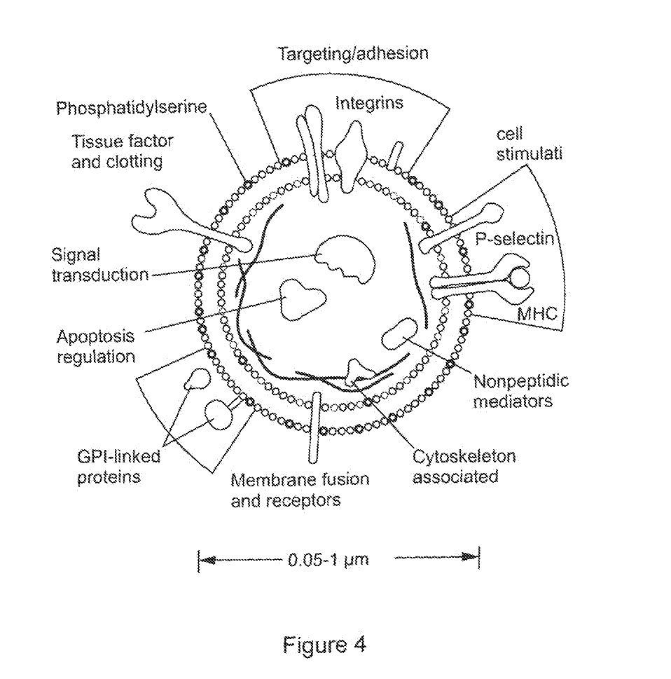

FIG. 5. Quantitation of Urinary Tissue Factor by ELISA in various cancers and normal individuals.

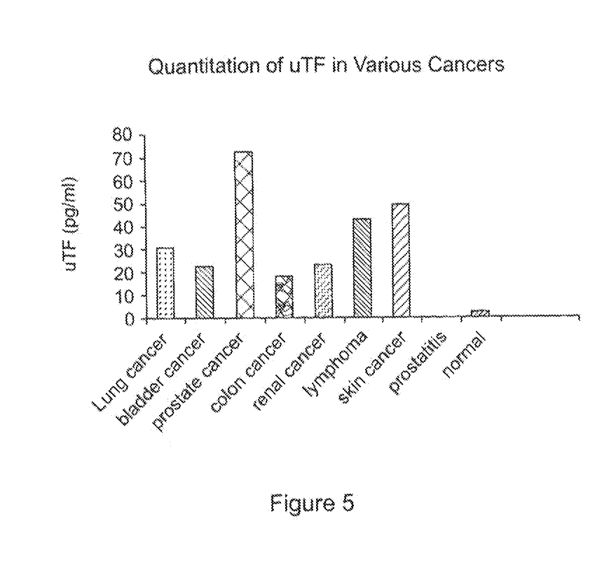

1. A method for detecting the c-terminal region (aka intracellular domain) of urinary tissue factor (uTF) in a sample comprising: a) binding an anti-uTF antibody against the c-terminal region of uTF to a solid phase; b) adding the sample to the solid phase, wherein uTF is present in the sample which binds to the antibody; c) adding an anti-uTF antibody to a different epitope in the c-terminal region than the capture antibody to the solid phase, wherein the uTF antibody binds to the c-terminal region of uTF, and d) detecting uTF by direct or indirect immunolabelling of the second anti-uTF antibody. The capture antibody can be against the amino acid sequence 275-285 within the c-terminal region of human Tissue factor and the detection antibody can be against the amino acid sequence 286-295 within the c-terminal region human Tissue factor.

2. The method of claim 1, wherein the sample is selected from the group consisting of a biological fluid, urine, saliva, whole blood, plasma, platelet rich plasma (PRP), platelet poor plasma (PPP), pooled normal plasma (PNP), and tissue culture supernatant.

3. The method of claim 1, wherein the solid phase is a membrane, plate, microwell or bead.

4. The method of claim 1, wherein detecting c-terminal region of uTF is by direct immunolabelling of the second c-terminal region anti-uTF antibody.

5. The method of claim 4, wherein the anti-uTF antibody is labeled with horse radish peroxidase.

6. The method of claim 1, wherein detecting c-terminal region of uTF is by indirect immunolabelling of the c-terminal region anti-uTF antibody.

7. The method of claim 6, wherein the anti-uTF antibody is raised in a first species and wherein a labeled antibody raised in a second species against antibodies from the first species is added to the solid phase and detected.

8. The method of claim 6, wherein the capture anti-uTF antibody is raised in a first species and wherein a labeled anti-uTF antibody raised in the same species as the first species is added to the solid phase and detected.

9. A method for measuring the amount of c-terminal region of uTF in a sample comprising the method of claim 6 and further comprising e) quantifying anti-uTF antibody detected, thereby measuring the amount of c-terminal region of uTF in the sample.

10. A method for diagnosing cancer in a subject comprising a) measuring the amount of uTF in a test sample from the subject according to the method of claim 8; and b) comparing the amount of c-terminal region of uTF in the test sample to the amount of c-terminal region of uTF in a control sample having normal c-terminal region of c-terminal region of uTF; wherein cancer is diagnosed by an increased amount of c-terminal region of uTF in the test sample compared with the control sample. The cancer can be solid cancers of various types and leukemia and lymphoma cancers of various types. The solid cancer can be but not limited to, breast, ovary, colon, central nervous system, kidney and prostate, kidney, bladder, breast, colorectal, liver, lung (non-small cell and small cell), brain, pancreas, stomach, esophagus and head and neck. The lymphoid cancer can be but not limited hodgkins lymphoma, non-hodgkins lymphoma, chronic lymphocytic leukemia, acute lymphocytic leukemia, chronic myeloid leukemia, acute myeloid leukemia.

11. The method of claim 10, wherein the test sample is selected from the group consisting of urine, saliva, whole blood, plasma, platelet rich plasma (PRP), platelet poor plasma (PPP), pooled normal plasma (PNP).

12. The method of claim 10, wherein the solid phase is a membrane, plate, microwell or bead.

13. The method of claim 10, wherein detecting c-terminal region of uTF is by direct immunolabelling of the anti-uTF antibody.

14. The method of claim 11, wherein the c-terminal region anti-uTF antibody is labeled with horse radish peroxidase.

15. The method of claim 14, wherein detecting c-terminal region of uTF is by indirect immunolabelling of the c-terminal region of anti-uTF antibody.

16. The method of claim 9, wherein the c-terminal region of anti-uTF antibody is raised in a first species and wherein a labeled antibody raised in a second species against antibodies from the first species is added to the solid phase and detected.

17. A kit for detecting c-terminal region of uTF in a sample comprising: (i) a solid phase coated with a c-terminal region anti-uTF antibody; and (ii) a second c-terminal region anti-uTF of antibody.

18. The kit of claim 17, wherein the second c-terminal region anti-uTF antibody is labeled.

19. The kit of claim 18, wherein the second c-terminal region anti-uTF antibody is labeled with horse radish peroxidase.

20. The kit of claim 17, further comprising a substrate for horse radish peroxidase.

21. The kit of claim 17, further comprising one or more components selected from the group consisting of a standard sample, a positive control and a wash buffer.
